(19) United States
(12) Patent Application Publication
(10) Pub. No.: US 2021/0322289 A1
(43) Pub. Date: Oct. 21, 2021

(54) SYNERGISTICALLY-ACTING COMPOSITIONS FOR MICROBE REDUCTION THAT COMPRISE AROMATIC ALCOHOL, GLYCERYL ETHER AND BISPYRIDINIUM ALKANE AND USE OF SUCH COMPOSITIONS

(71) Applicant: Schulke & Mayr GmbH, Norderstedt (DE)

(72) Inventors: Ralf GRADTKE, Norderstedt (DE); Sabine HERWEG, Norderstedt (DE); Stefan SAKULOWSKI, Norderstedt (DE); Klaus WEBER, Norderstedt (DE)

(21) Appl. No.: 17/272,099

(22) PCT Filed: Jul. 5, 2019
(86) PCT No.: PCT/EP2019/068154

§ 371 Date: Feb. 26, 2021

(30) Foreign Application Priority Data
Aug. 31, 2018 (DE) 10 2018 121 321.2

(51) Int. Cl. A61K 8/34; A61K 8/49

(52) U.S. Cl. CPC A61K 8/345; A61K 8/4926; A61K 2800/10; A61K 2800/70; A61K 2800/524; A61K 2800/5922

(57) ABSTRACT

The invention relates to synergistically-acting compositions for microbe reduction that comprise an aromatic alcohol, glyceryl ether and bispyridinium alkane. The invention also relates to the use of the synergistically-acting compositions, for example for microbe reduction and/or preservation of cosmetic compositions.

The invention relates to synergistically-acting compositions for microbe reduction that comprise an aromatic alcohol, glyceryl ether and bispyridinium alkane. The invention also relates to the use of synergistically-acting compositions, for example for microbe reduction and/or preservation of cosmetic compositions.

### BACKGROUND

To provide stability, preservatives are typically added to cosmetic compositions. Their primary purpose is to guarantee the long-term stability of the cosmetic composition, i.e. to ensure that the cosmetic composition remains free of microbial contamination for an adequately long period. However, it is also important that the preservative has fast-acting antimicrobial efficacy. On opening a container of a cosmetic composition, for example a pot or tube containing a cream, ointment, gel, powder or similar, there will, over the period of use, which may be anything from several months to years, depending on the nature of the cosmetic compositions, be repeated ingress of new microorganisms into the container, both completely unavoidably through contact with the ambient air, which inevitably contains microorganisms of all types, and particularly when the cosmetic composition is removed, which is typically done with the fingers or other aids that come into contact with the user's skin, for example brushes, wipes, cotton pads, etc. The greater the rapidity with which such microorganisms are killed after ingress, the easier it is to prevent permanent colonization of the cosmetic composition by microorganisms introduced in this manner.

To guarantee the stability of cosmetic compositions, they therefore usually have preservatives added. Although there is a relatively large selection of substances with potential preservative properties available for this to a person skilled in the art, for example organic acids, formaldehyde releasers, phenols, alcohols, parabens, aldehydes, isothiazolinones, organohalogen compounds, and quaternary ammonium compounds, the use of preservatives in cosmetic compositions is strictly regulated through EU Regulation No. 1223/2009, both in respect of whether preservatives are permitted at all and the maximum amounts in which their use is permitted. Thus, for example, the use of the aromatic alcohols phenoxyethanol and benzyl alcohol, which have been found to be perfectly suitable and are commonly used in the cosmetics field too, is limited by Annex (V) of this Regulation to a maximum concentration of 1.0% by weight in a cosmetic composition.

However, in many cosmetic formulations, this low concentration in which their use is permitted is insufficient for adequate preservation, particularly if the desired long-term efficacy and fast-acting effect are to be achieved at the same time. Moreover, since the non-fungicidal properties of aromatic alcohols in particular are often inadequate, aromatic alcohols are generally used in combination with other active substances. Of particular interest here are active substances and compounds that boost, i.e. intensify, the properties of the aromatic alcohols and in addition are ideally not subject to the restrictions of EU Regulation No. 1223/2009. One combination partner commonly used to boost aromatic alcohols is, for example, ethylhexylglycerin. A mixture of phenoxyethanol and ethylhexylglycerin is thus, for example, commercially available from Schülke & Mayr GmbH, Norderstedt (Germany) under the trade name euxyl PE 9010 for use as a preservative in cosmetic compositions.

However, for reasons associated with the formulation, it may be that even the booster combinations of aromatic alcohol and booster are not fast-acting enough. What is therefore needed is an improved booster system for aromatic alcohols that boosts the microbiological efficacy of the aromatic alcohol at the maximum content in which its use is permitted by Annex V of EU Regulation No. 1223/2009, for example 1% by weight in the case of both phenoxyethanol and benzyl alcohol, and achieves not only long-term efficacy but, in particular, a faster-acting effect.

### SUMMARY OF THE INVENTION

Surprisingly, it has been found that these and other objects are achieved by using a combination of an aromatic alcohol with a glyceryl ether and a bispyridinium alkane. The combination of the three components achieves an appreciably faster-acting and more pronounced antimicrobial effect than would be expected from the effect of each of the three components alone or of two-component combinations thereof. In particular, the microbiological efficacy of the combination of the three components is also demonstrated when used in a cosmetic composition in amounts in which the respective component alone does not show an adequate antimicrobial effect over the desired period. What was completely unexpected here is that the addition of bispyridinium alkane in amounts in which no microbiological/antimicrobial effect is observed for the bispyridinium alkane significantly boosts the antimicrobial effect of a combination of aromatic alcohol and glyceryl ether. The aromatic alcohol may likewise be present here in inactive amounts, i.e. in amounts in which the aromatic alcohol alone does not have an adequate microbiological/antimicrobial effect. The invention is thus based inter alia on the discovery of an unexpected synergistic effect when using the specific three-component combination of aromatic alcohol, glyceryl ether and bispyridinium alkane, with regard to its microbiological activity, particularly as regards its antimicrobial effect.

The invention therefore relates to a composition comprising
a) an aromatic alcohol,
b) a glyceryl ether, and
c) a bispyridinium alkane,

wherein the ratio by weight of component (c) to components (a) and (b) is 0.01 or less.

The invention also relates to the use of the composition according to the invention as described herein for the preservation of cosmetic formulations, for example rinse-off formulations or leave-on formulations.

Compositions according to the invention and preferred embodiments are described below in the detailed description and examples and also in the appended claims.

### DETAILED DESCRIPTION

In the description and the examples, all percentages relate to the total weight of the composition according to the invention. Percentages are generally percentages by weight unless stated expressly otherwise.

The compositions according to the invention comprise a) one or more aromatic alcohols. The aromatic alcohol may be selected from aryloxy alcohols, oligoalkanol aryl ethers and aryl alcohols and is preferably an aryloxy alcohol or aryl alcohol.

The aryloxy alcohols that may be used according to the invention have the general formula Ar—O—(CHR)ₙ—OH, wherein R is independently H (for n≥2) or C₁-C₆ alkyl and n is an integer, preferably an integer from 2 to 10, more preferably an integer from 2 to 6 and especially preferably 2 or 3.

The Ar group may be an aryl group that is substituted in the aromatic ring or unsubstituted, with an unsubstituted aryl group, for example phenyl or naphthyl, being preferred. Examples of aryloxy alkanols that may be used according to the invention are phenoxyethanol and phenoxypropanols, with the phenoxypropanols 1-phenoxypropan-2-ol, 2-phenoxypropan-1-ol or mixtures thereof and 3-phenoxypropan-1-ol being preferred.

Oligoalkanol aryl ethers include, for example, phenoxydiethanol, phenoxytriethanol and phenoxyoligoethanol and also phenoxydipropanol, phenoxytripropanol and phenoxyoligopropanol.

The aryl alkanols that may be used according to the invention have the general formula Ar—(CHR)ₙ—OH, in which R is independently H or C₁-C₆ alkyl and n is an integer, preferably from 1 to 10, more preferably an integer from 1 to 6 and especially preferably 1, 2, 3 or 4. The Ar group may be an aryl group that is substituted in the aromatic ring or unsubstituted, with an unsubstituted aryl group, for example phenyl or naphthyl, being preferred. Examples of aryl alkanols are 3-phenylpropan-1-ol, phenethyl alcohol, veratryl alcohol (3,4-dimethoxyphenylmethyl alcohol), benzyl alcohol and 2-methyl-1-phenyl-2-propanol.

The aromatic alcohol is preferably selected from the group consisting of phenoxyethanol, phenoxypropanol, phenylpropanol, phenethyl alcohol, veratryl alcohol, benzyl alcohol, 2-methyl-1-phenyl-2-propanol and combinations thereof. In an especially preferred embodiment of the present invention, the a) aromatic alcohol is selected from the group consisting of phenoxyethanol, phenethyl alcohol, benzyl alcohol and combinations thereof. Most preferred for the aromatic alcohol is phenoxyethanol.

The compositions according to the invention additionally comprise b) one or more glyceryl ethers.

The glyceryl ether may be a glyceryl monoalkyl ether, a glyceryl dialkyl ether, a glyceryl trialkyl ether or mixtures thereof, with the glyceryl ether being preferably a glyceryl monoalkyl ether. Examples of glyceryl monoalkyl ethers are glyceryl monoalkyl ethers that are substituted in the 1- or 2-position (i.e. asymmetrically or symmetrically; described hereinafter also as 1-alkyl glyceryl ethers or a 2-alkyl glyceryl ether), for example with a saturated or unsaturated, branched or unbranched alkyl residue.

In a preferred embodiment of the present invention, the b) glyceryl ether is a 1- or 2-(C₁-C₂₄alkyl) glyceryl ether, for example dodecyl glyceryl ether, octyl glyceryl ether, propyl glyceryl ether, octadecyl glyceryl ether (batyl alcohol), hexadecyl glyceryl ether (chimyl alcohol) and octadecenyl glyceryl ether (selachyl alcohol). Preferred are 1-monoalkyl glyceryl ethers that have a saturated branched or unbranched C₃-C₁₈ alkyl group, more preferred are 1-monoalkyl glyceryl ethers that have a saturated branched or unbranched C₆-C₁₂ alkyl group.

In an especially preferred embodiment of the present invention, the b) glyceryl ether is selected from the group consisting of ethylhexyl glyceryl ether, cyclohexyl glyceryl ether, caprylyl glyceryl ether and combinations thereof, most preferred is an ethylhexyl glyceryl ether, for example 1-(2-ethylhexyl) glyceryl ether (available as Sensiva® SC 50 from Schülke & Mayr GmbH, Norderstedt (Germany)).

The compositions according to the invention additionally comprise c) one or more bispyridinium alkanes.

In the composition according to the invention, all suitable bispyridinium alkanes may in principle be used. In a preferred embodiment of the present invention, the c) bispyridinium alkane is a bis[4-(substituted-amino)-1-pyridinium]alkane of general formula I or II,

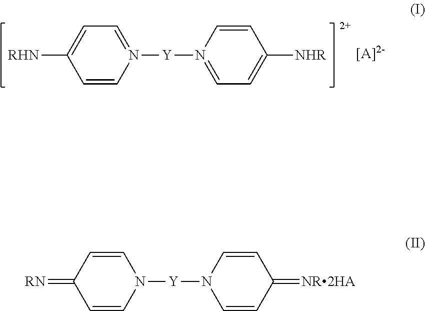

in which Y is an alkylene group with 4 to 18 carbon atoms, R is an alkyl group with 6 to 18 carbon atoms or a cycloalkyl group with 5 to 7 carbon atoms or a phenyl residue, which may be substituted by a halogen atom, and A is an anion or more than one anion. In an especially preferred embodiment, the bispyridinium alkane used according to the invention is N,N′-(1,10-decanediyldi-1[4H]-pyridinyl-4-ylidene)bis(1-octanamine) dihydrochloride (octenidine dihydrochloride (CAS No.: 70775-75-6), hereinafter also described as octenidine). Other octenidine salts besides this are also suitable.

In the compositions according to the invention, the ratio by weight of component c) a bispyridinium alkane to components a) an aromatic alcohol and b) a glyceryl ether is 0.01 or less. The expression “ratio by weight of component c) a bispyridinium alkane to components a) an aromatic alcohol and b) a glyceryl ether” as used here means the ratio of the proportion by weight of component c) to the sum of the proportions by weight of components a) and b) taken together. In other words, the ratio by weight of component c) a bispyridinium alkane to the sum of components a) an aromatic alcohol and b) a glyceryl ether is 0.01 or less. If the composition according to the invention comprises more than one component a) and/or more than one component b) and/or more than one component c), the proportions by weight of components a), b) and/or c) each relate to the total sum of the proportions by weight of the plurality in each case of components a), b) and/or c). The ratio of the proportion by weight of component c) to the sum of the proportions by weight of components a) and b) may also be expressed as 0.01:1 or less. The ratio by weight of component c) to components a) and b) is preferably 0.0001 to 0.01, more preferably 0.0001 to 0.005, and even more preferably 0.0005 to 0.002. These ratios by weight of component c) to components a) and b) may also be expressed as 0.0001:1 to 0.01:1, more preferably 0.0001:1 to 0.005:1, and even more preferably 0.0005:1 to 0.002:1.

In a preferred embodiment, the composition according to the invention thus comprises
a) an aromatic alcohol, which is an aryloxy alkanol or an aryl alkanol,
b) a glyceryl ether, which is selected from the group consisting of ethylhexyl glyceryl ether, cyclohexyl glyceryl ether, caprylyl glyceryl ether and combinations thereof, and
c) a bispyridinium alkane, preferably octenidine,

wherein the ratio by weight of component c) to components a) and b) is 0.0001 to 0.01, preferably 0.0001 to 0.005, more preferably 0.0005 to 0.002.

In a further preferred embodiment, the composition according to the invention comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof,
b) a glyceryl ether, which is selected from the group consisting of ethylhexyl glyceryl ether, cyclohexyl glyceryl ether, caprylyl glyceryl ether and combinations thereof, and
c) a bispyridinium alkane, preferably octenidine,

wherein the ratio by weight of component c) to components a) and b) is 0.0001 to 0.01.

In a further preferred embodiment, the composition according to the invention comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof,
b) a glyceryl ether, which is selected from the group consisting of ethylhexyl glyceryl ether, cyclohexyl glyceryl ether, caprylyl glyceryl ether and combinations thereof, and
c) a bispyridinium alkane, preferably octenidine,

wherein the ratio by weight of component c) to components a) and b) is 0.0001 to 0.005, preferably 0.0005 to 0.002.

In a further preferred embodiment, the composition according to the invention comprises
a) an aromatic alcohol, selected from the group consisting of phenoxyethanol, phenethyl alcohol, benzyl alcohol and combinations thereof,
b) a glyceryl ether, which is selected from the group consisting of ethylhexyl glyceryl ether, cyclohexyl glyceryl ether, caprylyl glyceryl ether and combinations thereof, and
c) a bispyridinium alkane, preferably octenidine,

wherein the ratio by weight of component c) to components a) and b) is 0.0001 to 0.005, preferably 0.0005 to 0.002.

In an especially preferred embodiment, the composition according to the invention comprises
a) an aromatic alcohol, selected from the group consisting of phenoxyethanol, phenethyl alcohol, benzyl alcohol and combinations thereof,
b) a glyceryl ether, which is ethylhexyl glyceryl ether, and
c) a bispyridinium alkane, which is octenidine,

wherein the ratio by weight of component c) to components a) and b) is 0.0005 to 0.002.

In another embodiment, the composition according to the invention may also comprise
a) an aromatic alcohol, selected from the group consisting of phenoxyethanol, phenethyl alcohol, benzyl alcohol and combinations thereof,
b) a glyceryl ether, which is ethylhexyl glyceryl ether, and
c) a bispyridinium alkane, which is octenidine,

wherein the ratio by weight of component c) to components a) and b) is 0.0001 to 0.01, preferably 0.0001 to 0.005, more preferably 0.0005 to 0.002.

The ratio of component b) a glyceryl ether to component a) an aromatic alcohol is in principle unrestricted. However, in a further preferred embodiment of the composition according to the invention, the ratio by weight of component b) a glyceryl ether to component a) an aromatic alcohol is additionally 0.3 or less, for example 0.01 to 0.3 or 0.01 to 0.2, and especially preferably 0.05 to 0.15. Here too, the ratio by weight of component b) a glyceryl ether to component a) an aromatic alcohol may be expressed as 0.3:1 or less, for example from 0.01:1 to 0.3:1 or 0.01:1 to 0.2:1, especially preferably 0.05:1 to 0.15:1. If the composition according to the invention comprises more than one component a) and/or more than one component b), the proportions by weight of components a) and/or b) each relate to the total sum of the proportions by weight of the plurality in each case of components a) and/or b).

The composition according to the invention may contain additional components, for example further active substances, solvents, and/or further added substances/excipients/additives, for example antioxidants.

The composition according to the invention may take the form of a concentrate that, during production of for example cosmetic compositions, is added to the latter in sufficient amount to provide stability.

If the composition according to the invention is in the form of a concentrate, the composition comprises
a) the aromatic alcohol typically in an amount from 50 to 98.99% by weight, preferably from 80 to 98.99% by weight,
b) the glyceryl ether typically in an amount from 1 to 30% by weight, preferably from 1 to 20% by weight, and
c) the bispyridinium alkane typically in an amount from 0.01 to 1% by weight, preferably from 0.05 to 0.5% by weight.

The composition according to the invention, if it is in the form of a concentrate, thus comprises in a preferred embodiment
a) an aromatic alcohol, which is an aryloxy alkanol or an aryl alkanol, in an amount from 50 to 98.99% by weight, preferably from 80 to 98.99% by weight,
b) a glyceryl ether, selected from the group consisting of ethylhexyl glyceryl ether, cyclohexyl glyceryl ether, caprylyl glyceryl ether and combinations thereof, in an amount from 1 to 30% by weight, preferably from 1 to 20% by weight, and
c) a bispyridinium alkane, preferably octenidine, in an amount from 0.01 to 1% by weight, preferably from 0.05 to 0.5% by weight.

wherein the ratio by weight of component c) to components a) and b) is 0.0001 to 0.01, preferably 0.0001 to 0.005, more preferably 0.0005 to 0.002.

In a further preferred embodiment, the composition according to the invention, if it is in the form of a concentrate, comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof, in an amount from 50 to 98.99% by weight,
b) a glyceryl ether, selected from the group consisting of ethylhexyl glyceryl ether, cyclohexyl glyceryl ether, caprylyl glyceryl ether and combinations thereof, in an amount from 1 to 30% by weight, and
c) a bispyridinium alkane, preferably octenidine, in an amount from 0.01 to 1% by weight.

wherein the ratio by weight of component c) to components a) and b) is 0.0001 to 0.01.

In a further preferred embodiment, the composition according to the invention, if it is in the form of a concentrate, comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof, in an amount from 80 to 98.99% by weight,
b) a glyceryl ether, selected from the group consisting of ethylhexyl glyceryl ether, cyclohexyl glyceryl ether, caprylyl glyceryl ether and combinations thereof, in an amount from 1 to 20% by weight, and
c) a bispyridinium alkane, preferably octenidine, in an amount from 0.05 to 0.5% by weight.

wherein the ratio by weight of component c) to components a) and b) is 0.0001 to 0.005.

In a further preferred embodiment, the composition according to the invention, if it is in the form of a concentrate, comprises
a) an aromatic alcohol, selected from the group consisting of phenoxyethanol, phenethyl alcohol, benzyl alcohol and combinations thereof, in an amount from 80 to 98.99% by weight,
b) a glyceryl ether, selected from the group consisting of ethylhexyl glyceryl ether, cyclohexyl glyceryl ether, caprylyl glyceryl ether and combinations thereof, in an amount from 1 to 20% by weight, and
c) a bispyridinium alkane, preferably octenidine, in an amount from 0.05 to 0.5% by weight.

wherein the ratio by weight of component c) to components a) and b) is 0.0001 to 0.005.

In an especially preferred embodiment, the composition according to the invention, if it is in the form of a concentrate, comprises
a) an aromatic alcohol, selected from the group consisting of phenoxyethanol, phenethyl alcohol, benzyl alcohol and combinations thereof, in an amount from 80 to 98.99% by weight,
b) a glyceryl ether, which is ethylhexyl glyceryl ether, in an amount from 1 to 20% by weight, and
c) a bispyridinium alkane, which is octenidine, in an amount from 0.05 to 0.5% by weight.

wherein the ratio by weight of component c) to components a) and b) is 0.0005 to 0.002.

In another embodiment, the composition according to the invention, if it is in the form of a concentrate, may also comprise
a) an aromatic alcohol, selected from the group consisting of phenoxyethanol, phenethyl alcohol, benzyl alcohol and combinations thereof, in an amount from 80 to 98.95% by weight,
b) a glyceryl ether, which is ethylhexyl glyceryl ether, in an amount from 1 to 20% by weight, and
c) a bispyridinium alkane, which is octenidine, in an amount from 0.05 to 0.5% by weight.

wherein the ratio by weight of component c) to components a) and b) is 0.0001 to 0.01, preferably 0.0001 to 0.005, more preferably 0.0005 to 0.002 and wherein the sum of components a), b) and c) is 100% by weight.

If the composition according to the invention is in the form of a concentrate, there may also in principle be further components present in the concentrate, for example stabilizers and antioxidants. The concentrate should, however, normally comprise a total of not less than 85% by weight, preferably not less than 90% by weight, more preferably not less than 95% by weight, and especially preferably not less than 98% by weight of components a), b) and c), relative to the total weight of the concentrate.

The composition according to the invention may also take the form of a cosmetic composition that, in addition to the usual components of such compositions, includes the components a) an aromatic alcohol, b) a glyceryl ether, and c) a bispyridinium alkane. Such cosmetic compositions may in principle be any cosmetic composition or formulation, for example creams, ointments, powders, make-up, body lotions, hairsprays, shampoos, moist wipes. The respective cosmetic compositions may contain components selected from all common components of cosmetic compositions familiar to a person skilled in the art. The three-component combination of aromatic alcohol, glyceryl ether and bispyridinium alkane described herein is suitable in principle for microbe reduction in a large number of cosmetic compositions.

If the composition according to the invention is in the form of a cosmetic composition, the composition comprises
a) the aromatic alcohol typically in an amount from 0.01 to 3% by weight, preferably from 0.1 to 2.0% by weight, more preferably from 0.5 to 1.5% by weight,
b) the glyceryl ether typically in an amount from 0.001 to 0.5% by weight, preferably from 0.01 to 0.2% by weight,
c) the bispyridinium alkane typically in an amount from 0.00001 to 0.01% by weight (corresponding to 0.1 ppm to 100 ppm, relative to the total weight of the cosmetic composition), preferably from 0.0001 to 0.002% by weight (corresponding to 1 ppm to 20 ppm, relative to the total weight of the cosmetic composition) and more preferably from 0.0005 to 0.002% by weight (corresponding to 5 ppm to 20 ppm, relative to the total weight of the cosmetic composition).

The composition according to the invention, if it is in the form of a cosmetic composition, thus comprises in a preferred embodiment
a) an aromatic alcohol, which is an aryloxy alkanol or an aryl alkanol, in an amount from 0.01 to 30% by weight,
b) a glyceryl ether, selected from the group consisting of ethylhexyl glyceryl ether, cyclohexyl glyceryl ether, caprylyl glyceryl ether and combinations thereof, in an amount from 0.001 to 0.5% by weight, and
c) a bispyridinium alkane, preferably octenidine, in an amount from 0.00001 to 0.01% by weight.

wherein the ratio by weight of component c) to components a) and b) is 0.0001 to 0.01, preferably 0.0001 to 0.005, more preferably 0.0005 to 0.002.

In a further preferred embodiment, the composition according to the invention, if it is in the form of a cosmetic composition, comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof, in an amount from 0.01 to 3% by weight,
b) a glyceryl ether, selected from the group consisting of ethylhexyl glyceryl ether, cyclohexyl glyceryl ether, caprylyl glyceryl ether and combinations thereof, in an amount from 0.001 to 0.5% by weight, and
c) a bispyridinium alkane, preferably octenidine, in an amount from 0.00001 to 0.01% by weight.

wherein the ratio by weight of component c) to components a) and b) is 0.0001 to 0.01.

In a further preferred embodiment, the composition according to the invention, if it is in the form of a cosmetic composition, comprises
a) an aromatic alcohol, selected from the group consisting of phenoxyethanol, phenoxypropanol, phenylpropanol, phenethyl alcohol, veratryl alcohol, benzyl alcohol, 2-methyl-1-phenyl-2-propanol and combinations thereof, in an amount from 0.1 to 2.0% by weight, preferably in an amount from 0.5 to 1.5% by weight,
b) a glyceryl ether, selected from the group consisting of ethylhexyl glyceryl ether, cyclohexyl glyceryl ether, caprylyl glyceryl ether and combinations thereof, in an amount from 0.01 to 0.2% by weight, and
c) a bispyridinium alkane, preferably octenidine, in an amount from 0.0001 to 0.002% by weight, preferably in an amount from 0.0005 to 0.002% by weight.

wherein the ratio by weight of component c) to components a) and b) is 0.0001 to 0.005.

In a further preferred embodiment, the composition according to the invention, if it is in the form of a cosmetic composition, comprises
a) an aromatic alcohol, selected from the group consisting of phenoxyethanol, phenethyl alcohol, benzyl alcohol and combinations thereof, in an amount from 0.1 to 2.0% by weight, preferably in an amount from 0.5 to 1.5% by weight,
b) a glyceryl ether, selected from the group consisting of ethylhexyl glyceryl ether, cyclohexyl glyceryl ether, caprylyl glyceryl ether and combinations thereof, in an amount from 0.01 to 0.2% by weight, and
c) a bispyridinium alkane, preferably octenidine, in an amount from 0.0001 to 0.002% by weight, preferably in an amount from 0.0005 to 0.002% by weight.

wherein the ratio by weight of component c) to components a) and b) is 0.0001 to 0.005.

In an especially preferred embodiment, the composition according to the invention, if it is in the form of a cosmetic composition, comprises
a) an aromatic alcohol, selected from the group consisting of phenoxyethanol, phenethyl alcohol, benzyl alcohol and combinations thereof, in an amount from 0.1 to 2.0% by weight, preferably in an amount from 0.5 to 1.5% by weight,
b) a glyceryl ether, which is ethylhexyl glyceryl ether, in an amount from 0.01 to 0.2% by weight, and
c) a bispyridinium alkane, which is octenidine, in an amount from 0.0001 to 0.002% by weight, preferably in an amount from 0.0005 to 0.002% by weight.

wherein the ratio by weight of component c) to components a) and b) is 0.0005 to 0.002.

The invention also relates to the use of the composition according to the present invention as described herein, particularly when it is in the form of a concentrate, for microbe reduction and/or preservation of cosmetic formulations, for example rinse-off formulations or leave-on formulations. Rinse-off formulations are cosmetic products that are rinsed off after a brief contact time and include, for example, shower gels, shampoos, liquid soaps, hair conditioners, etc. Leave-on formulations are cosmetic products that remain on the skin or hair and include, for example, creams, ointments, powders, make-up, body lotions, hairsprays, styling gels, moist wipe applications, etc. The use of the composition according to the invention, particularly when it is in the form of a concentrate, is not restricted to particular cosmetic compositions, i.e. the composition according to the invention may in principle be used in any cosmetic compositions.

The advantages of the present invention are also particularly apparent from the following examples.

### Examples

The improved microbiological/antimicrobial efficacy of the compositions according to the invention has been demonstrated by a microbial reduction test.

The microbial reduction tests described below were performed using commercially available phenylpropanol, phenoxyethanol, benzyl alcohol, phenethyl alcohol, 2-methyl-1-phenyl-2-propanol, cyclohexyl glyceryl ether, caprylyl glyceryl ether, ethylhexylglycerin and octenidine dihydrochloride and the commercially available mixtures of phenoxyethanol and ethylhexylglycerin (90:10, obtained as euxyl PE 9010 from Schülke & Mayr GmbH, Norderstedt (Germany)), of benzyl alcohol and ethylhexylglycerin (90:10, obtained as euxyl K 900 from Schülke & Mayr GmbH, Norderstedt (Germany))) and of phenethyl alcohol and ethylhexylglycerin (90:10, obtained as sensiva PA 20 from Schulke & Mayr GmbH, Norderstedt). All components were used as obtained, except for octenidine dihydrochloride, which was used as a 0.25% by weight solution in deionized water prepared in an initial step.

The microbial reduction tests were performed using the strain Aspergillus brasiliensis ATCC 16404. The test microorganism was cultured on Sabouraud agar plates for seven days at 25° C. The culture was then washed off with physiological saline (0.85% w/v) and Triton X-100 (3-5 drops) and filtered through a glass wool filter. The spore suspension was adjusted to approx. 107 CFU/ml (CFU=colony-forming units) with physiological saline. In each test, 25 g of the test substance was inoculated with 0.1 ml of of the spore suspension (microbial count in the test matrix approx. 105 CFU/g) and stirred thoroughly. After selected times, a sample was withdrawn and streaked onto Sabouraud agar containing disinhibitors (addition of 3% Tween 80, 0.3% lecithin, 3% saponin, 0.1% histidine). The streaks were incubated for 72 h at 25° C. The growth of the streaks was then evaluated semiquantitatively. The observed growth is evaluated as follows: −: free of microbial growth; +: slight growth; ++: moderate growth; +++: strong growth; ++++: very strong growth; R: completely covered.

The tests are summarized in Tables 1 and 2 below. All % values shown are percentages by weight relative to the total weight of the composition tested in each case. Similarly, the ppm values shown are proportions by weight relative to the total weight of the composition tested in each case. Octenidine stands for octenidine dihydrochloride.

TABLE 1

| | | Growth of Aspergillus brasiliensis | | | | |
|---|---|---|---|---|---|---|
| Test | | after contact time in [h] | | | | |
| No. | Test material/Product | 0 h | 6 h | 24 h | 48 h | 72 h |
| 1 | blank; deionized water | R | R | R | R | R |
| 2 | +5 ppm octenidine | R | R | R | R | R |
| 3 | +10 ppm octenidine | R | R | R | R | R |
| 4 | +15 ppm octenidine | R | R | R | R | R |
| 5 | +0.05% ethylhexyl- | R | R | R | R | R |
| | glycerin (sensiva SC 50) | | | | | |
| 6 | +0.10% ethylhexyl- | R | R | R | R | R |
| | glycerin (sensiva SC 50) | | | | | |
| 7 | +0.15% ethylhexyl- | R | R | R | R | R |
| | glycerin (sensiva SC 50) | | | | | |
| 8 | +0.10% cyclohexyl | R | R | R | R | R |
| | glyceryl ether | | | | | |
| 9 | +0.10% caprylyl | R | R | R | R | R |
| | glyceryl ether | | | | | |
| 10 | +1.00% phenoxyethanol | R | R | R | R | R |
| 11 | +1.00% benzyl alcohol | R | R | R | R | R |
| 12 | +1.00% phenethyl | R | R | R | − | − |
| | alcohol | | | | | |
| 13 | +1.00% 2-methyl-1- | R | R | R | R | R |
| | phenyl-2-propanol | | | | | |
| 14 | +5 ppm octenidine + | R | R | R | R | R |
| | 0.05% ethylhexyl- | | | | | |
| | glycerin (sensiva SC 50) | | | | | |
| 15 | +10 ppm octenidine + | R | R | R | R | R |
| | 0.05% ethylhexyl- | | | | | |
| | glycerin (sensiva SC 50) | | | | | |
| 16 | +15 ppm octenidine + | R | R | R | R | R |
| | 0.05% ethylhexyl- | | | | | |
| | glycerin (sensiva SC 50) | | | | | |
| 17 | +10 ppm octenidine + | R | R | R | R | R |
| | 0.10% ethylhexyl- | | | | | |
| | glycerin (sensiva SC 50) | | | | | |
| 18 | +15 ppm octenidine + | R | R | R | R | R |
| | 0.10% ethylhexyl- | | | | | |
| | glycerin (sensiva SC 50) | | | | | |
| 19 | +15 ppm octenidine + | R | R | R | R | R |
| | 0.15% ethylhexyl- | | | | | |
| | glycerin (sensiva SC 50) | | | | | |
| 20 | +1.00% phenoxy- | R | R | R | +++ | ++ |
| | ethanol + 5 ppm | | | | | |
| | octenidine | | | | | |
| 21 | +1.00% phenoxy- | R | R | R | +++ | ++ |
| | ethanol + 10 ppm | | | | | |
| | octenidine | | | | | |
| 22 | +1.00% phenoxy- | R | R | R | +++ | ++ |
| | ethanol + 15 ppm | | | | | |
| | octenidine | | | | | |
| 23 | +0.90% phenoxy- | R | R | R | R | R |
| | ethanol + 0.10% | | | | | |
| | ethylhexylglycerin (added | | | | | |
| | together as 1.00% euxyl | | | | | |
| | PE 9010) | | | | | |
| 24 | +0.90% phenoxy- | R | R | R | + | − |
| | ethanol + 0.10% | | | | | |
| | ethylhexylglycerin | | | | | |
| | (added together as 1.00% | | | | | |
| | euxyl PE 9010) + 5 ppm | | | | | |
| | octenidine | | | | | |
| 25 | +0.90% phenoxyethanol + | R | R | R | + | − |
| | 0.10% ethylhexyl- | | | | | |
| | glycerin (added together | | | | | |
| | as 1.00% euxyl PE | | | | | |
| | 9010) + 10 ppm | | | | | |
| | octenidine | | | | | |
| 26 | +0.90% phenoxy- | R | R | R | + | − |
| | ethanol + 0.10% | | | | | |
| | ethylhexylglycerin | | | | | |
| | (added together as | | | | | |
| | 1.00% euxyl PE 9010) + | | | | | |
| | 15 ppm octenidine | | | | | |
| 27 | +0.99% phenoxy- | R | R | R | ++ | + |
| | ethanol + 0.01% | | | | | |
| | ethylhexylglycerin + | | | | | |
| | 10 ppm octenidine | | | | | |
| 28 | +0.99% phenoxy- | R | R | R | ++ | + |
| | ethanol + 0.01% | | | | | |
| | ethylhexylglycerin + | | | | | |
| | 15 ppm octenidine | | | | | |
| 29 | +1.00% benzyl alcohol + | R | R | R | ++++ | + |
| | 10 ppm octenidine | | | | | |
| 30 | +1.00% benzyl alcohol + | R | R | R | ++++ | + |
| | 15 ppm octenidine | | | | | |
| 31 | +0.90% benzyl alcohol + | R | R | R | R | R |
| | 0.10% ethylhexyl- | | | | | |
| | glycerin (added together | | | | | |
| | as euxyl K 900) | | | | | |
| 32 | +0.90% benzyl alcohol + | R | R | R | + | + |
| | 0.10% ethylhexyl- | | | | | |
| | glycerin (added together | | | | | |
| | as euxyl K | | | | | |
| | 900) + 5 ppm octenidine | | | | | |
| 33 | 0.90% benzyl alcohol + | R | R | R | + | + |
| | 0.10% ethylhexyl- | | | | | |
| | glycerin (added together | | | | | |
| | as euxyl | | | | | |
| | K 900) + 10 ppm | | | | | |
| | octenidine | | | | | |
| 34 | +0.90% benzyl alcohol + | R | R | R | + | + |
| | 0.10% ethylhexyl- | | | | | |
| | glycerin (added together | | | | | |
| | as euxyl | | | | | |
| | K 900) + 15 ppm | | | | | |
| | octenidine | | | | | |
| 35 | +0.80% benzyl alcohol + | R | R | R | ++ | − |
| | 0.20% ethylhexylglycerin | | | | | |
| 36 | +0.80% benzyl alcohol + | R | R | +++ | − | − |
| | 0.20% ethylhexyl- | | | | | |
| | glycerin + 5 ppm | | | | | |
| | octenidine | | | | | |
| 37 | +0.80% benzyl alcohol + | R | R | +++ | − | − |
| | 0.20% ethylhexyl- | | | | | |
| | glycerin + 10 ppm | | | | | |
| | octenidine | | | | | |
| 38 | +0.80% benzyl alcohol + | R | R | +++ | − | − |
| | 0.20% ethylhexyl- | | | | | |
| | glycerin + 15 ppm | | | | | |
| | octenidine | | | | | |
| 39 | +0.99% benzyl alcohol + | R | R | R | R | R |
| | 0.01% ethylhexylglycerin | | | | | |
| 40 | +0.99% benzyl alcohol + | R | R | R | ++++ | ++ |
| | 0.01% ethylhexyl- | | | | | |
| | glycerin + 5 ppm | | | | | |
| | octenidine | | | | | |
| 41 | +0.99% benzyl alcohol + | R | R | R | +++ | + |
| | 0.01% ethylhexyl- | | | | | |
| | glycerin + 10 ppm | | | | | |
| | octenidine | | | | | |
| 42 | +0.99% benzyl alcohol + | R | R | R | +++ | + |
| | 0.01% ethylhexyl- | | | | | |
| | glycerin + 15 ppm | | | | | |
| | octenidine | | | | | |
| 43 | +1.00% phenethyl | R | R | +++ | − | − |
| | alcohol + 10 ppm | | | | | |
| | octenidine | | | | | |
| 44 | +0.90% phenethyl | R | R | R | − | − |
| | alcohol + 0.10% ethyl- | | | | | |
| | hexylglycerin (added | | | | | |
| | together as sensiva PA 20) | | | | | |
| 45 | +0.90% phenethyl | R | R | ++ | − | − |
| | alcohol + 0.10% ethyl- | | | | | |
| | hexylglycerin (added | | | | | |
| | together as sensiva PA | | | | | |
| | 20) + 10 ppm octenidine | | | | | |
| 46 | +0.99% phenethyl | R | R | R | − | − |
| | alcohol + 0.01% ethyl- | | | | | |
| | hexylglycerin | | | | | |
| 47 | +0.99% phenethyl | R | R | ++ | − | − |
| | alcohol + 0.01% ethyl- | | | | | |
| | hexylglycerin + 10 ppm | | | | | |
| | octenidine | | | | | |
| 48 | +0.80% phenethyl | R | R | ++ | − | − |
| | alcohol + 0.20% | | | | | |
| | ethylhexylglycerin | | | | | |
| 49 | +0.80% phenethyl | R | R | + | − | − |
| | alcohol + 0.20% ethyl- | | | | | |
| | hexylglycerin + 10 ppm | | | | | |
| | octenidine | | | | | |
| 50 | +0.10% cyclohexyl | R | R | R | R | R |
| | glyceryl ether + | | | | | |
| | 10 ppm octenidine | | | | | |
| 51 | +1.00% phenoxy- | R | R | R | R | R |
| | ethanol + 0.10% | | | | | |
| | cyclohexyl glyceryl ether | | | | | |
| 52 | +1.00% phenoxy- | R | R | ++++ | ++ | + |
| | ethanol + 0.10% | | | | | |
| | cyclohexyl glyceryl | | | | | |
| | ether + 10 ppm octenidine | | | | | |
| 53 | +0.10% caprylyl glyceryl | R | R | R | R | R |
| | ether + 10 ppm octenidine | | | | | |
| 54 | +1.00% phenoxy- | R | R | R | ++ | − |
| | ethanol + 0.10% caprylyl | | | | | |
| | glyceryl ether | | | | | |
| 55 | +1.00% phenoxyethanol + | R | R | +++ | + | − |
| | 0.10% caprylyl glyceryl | | | | | |
| | ether + 10 ppm octenidine | | | | | |
| 56 | +1.00% benzyl alcohol + | R | R | R | R | R |
| | 0.10% cyclohexyl | | | | | |
| | glyceryl ether | | | | | |
| 57 | +1.00% benzyl alcohol + | R | R | R | +++ | + |
| | 0.10% cyclohexyl | | | | | |
| | glyceryl ether + | | | | | |
| | 10 ppm octenidine | | | | | |
| 58 | +1.00% benzyl alcohol + | R | R | R | + | − |
| | 0.10% caprylyl glyceryl | | | | | |
| | ether | | | | | |
| 59 | +1.00% benzyl alcohol + | R | R | ++++ | − | − |
| | 0.10% caprylyl glyceryl | | | | | |
| | ether + 10 ppm octenidine | | | | | |
| 60 | +1.00% phenethyl | R | R | R | R | |
| | alcohol + 0.10% cyclo- | | | | | |
| | hexyl glyceryl ether | | | | | |
| 61 | +1.00% phenethyl | R | R | ++++ | − | |
| | alcohol + 0.10% | | | | | |
| | cyclohexyl glyceryl | | | | | |
| | ether + 10 ppm octenidine | | | | | |
| 62 | +0.90% 2-methyl-1- | R | R | R | ++ | + |
| | phenyl-2-propanol + | | | | | |
| | 0.10% ethylhexyl- | | | | | |
| | glycerin | | | | | |
| 63 | +0.90% 2-methyl-1- | R | R | R | − | − |
| | phenyl-2-propanol + | | | | | |
| | 0.10% ethylhexyl- | | | | | |
| | glycerin + 10 ppm | | | | | |
| | octenidine | | | | | |

TABLE 2

| | | Growth of Aspergillus brasiliensis after | | | |
|---|---|---|---|---|---|
| | | contact time in [h] | | | |
| Test No. | | 0 h | 6 h | 12 h | 24 h |
| 64 | + 1.00% phenylpropanol | R | R | R | − |
| 65 | + 1.00% phenylpropanol | R | R | ++ | − |
| | + 0.10% caprylyl glyceryl ether | | | | |
| 66 | + 1.00% phenylpropanol | R | +++ | + | − |
| | + 0.10% caprylyl glyceryl ether | | | | |
| | + 10 ppm octenidine | | | | |

The three-component synergism between a) an aromatic alcohol, b) a glyceryl ether and c) a bispyridinium alkane as regards antimicrobial effect can be seen clearly from Tables 1 and 2 above. Tests Nos. 2-13 and 64 were performed for various individual components, in some cases in varying amounts too. With the exception of phenethyl alcohol (test No. 12) and phenylpropanol (test No. 64), no microbe reduction was observed for any of the individual components, even after 72 hours. In other words, the respective components on their own can be considered to have been used in ineffective amounts. Many two-component combinations of the three possible components likewise show no microbe reduction (see tests Nos. 14-19, 23, 31, 39, 50, 51, 53, 56 and 60). On the other hand, in all cases the three-component combination of components in the composition according to the invention has a considerably stronger antimicrobial effect than would have been expected from the effect of the individual components or two-component combinations. This is the case even when a certain antimicrobial effect had already been demonstrated for various two-component combinations (see tests Nos. 20-22, 29, 30, 43, 46, 48, 54, 58 and 62). On addition of the third component of the composition according to the invention, a further significant improvement in antimicrobial effect was in all cases observed. The improvement is a consequence primarily of the ability of the compositions according to the invention to reduce the microbial count in the compositions used as examples within just a short space of time (usually after 18 hours, but sometimes after just 6 or 12 hours) and in all cases within a considerably earlier time than the respective two-component combinations. It should also be noted here that the two-component combinations used in tests 23 and 31 are compositions currently used to provide stability in cosmetic compositions.

1. A composition comprising:
a) an aromatic alcohol,
b) a glyceryl ether, and
c) a bispyridinium alkane,
wherein the ratio by weight of component c) to components a) and b) is 0.01 or less.

2. The composition according to claim 1, wherein the bispyridinium alkane is an octenidine salt, preferably octenidine dihydrochloride.

3. The composition according to claim 1, wherein the aromatic alcohol is an aryloxy alkanol or an aryl alkanol.

4. The composition according to claim 3, wherein the aryloxy alkanol is selected from the group consisting of phenoxyethanol, phenoxypropanol and combinations thereof and the aryl alkanol is selected from the group consisting of phenylpropanol, phenethyl alcohol, veratryl alcohol, benzyl alcohol, 2-methyl-1-phenyl-2-propanol and combinations thereof.

5. The composition according to claim 1, wherein the aromatic alcohol is selected from the group consisting of phenoxyethanol, phenethyl alcohol, benzyl alcohol and combinations thereof.

6. The composition according to claim 1, wherein the glyceryl ether is a I-or 2-(C1-C24 alkyl) glyceryl ether.

7. The composition according to claim 6, wherein the glyceryl ether is a 1-monoalkyl glyceryl ether that has a saturated branched or unbranched C3-C1s alkyl group.

8. The composition according to claim 1, wherein the glyceryl ether is selected from the group consisting of ethylhexyl glyceryl ether, cyclohexyl glyceryl ether, caprylyl glyceryl ether and combinations thereof, and preferably is ethylhexyl glyceryl ether.

9. The composition according to claim 1, wherein the ratio by weight of component c) to components a) and b) is 0.0001 to 0.01, preferably 0.0001 to 0.005, more preferably 0.0005 to 0.002.

10. The composition according to claim 1, wherein the ratio by weight of component b) to component a) is 0.3 or less, preferably 0.01 to 0.3, more preferably 0.01 to 0.2, even more preferably 0.05 to 0.15.

11. The composition according to claim 1, comprising:
a) the aromatic alcohol in an amount from 50 to 98.99% by weight, preferably from 80 to 99% by weight,
b) the glyceryl ether in an amount from 1 to 30% by weight, preferably from 1 to 20% by weight, and
c) the bispyridinium alkane in an amount from 0.01 to 1% by weight, preferably from 0.05 to 0.5% by weight.

12. The composition according to claim 1, comprising:
a) the aromatic alcohol in an amount from 0.01 to 3% by weight, preferably from 0.1 to 2.0% by weight, more preferably 0.5 to 1.5% by weight,
b) the glyceryl ether in an amount from 0.001 to 0.5% by weight, preferably from 0.01 to 0.2% by weight, and
c) the bispyridinium alkane in an amount from 0.00001 to 0.01% by weight, preferably from 0.0001 to 0.002% by weight, more preferably from 0.0005 to 0.002% by weight.

13. The use of the composition according to claim 1 for microbe reduction and/or preservation of cosmetic formulations, for example rinse-off formulations or leave-on formulations, preferably leave-on formulations.

14. The use of the composition according to claim 11 for microbe reduction and/or preservation of cosmetic formulations, for example rinse-off formulations or leave-on formulations, preferably leave-on formulations.

15. The use of the composition according to claim 12 for microbe reduction and/or preservation of cosmetic formulations, for example rinse-off formulations or leave-on formulations, preferably leave-on formulations.
